(19) United States
(12) Patent Application Publication
(10) Pub. No.: US 2008/0214530 A1
(43) Pub. Date: Sep. 4, 2008

(54) Photo-Activated Disinfection

(76) Inventor: Michael John Colles, London (GB)

Correspondence Address: CONNOLLY BOVE LODGE & HUTZ LLP, 1875 EYE STREET, N.W., SUITE 1100, WASHINGTON, DC, 20036, US

(21) Appl. No.: 10/558,026

(22) PCT Filed: May 21, 2004
(86) PCT No.: PCT/GB04/02187

§ 371 Date: Nov. 28, 2007

(30) Foreign Application Priority Data
May 23, 2003 (GB) 0311950.0

(51) Int. Cl. A61K 31/5415; A61P 31/00; A61C 3/00

(52) U.S. Cl. 514/224.8; 433/29

(57) ABSTRACT

The present invention relates to photo-activated disinfection. In particular, the present invention relates to photo-activated disinfection in the oral cavity. We describe a dental apparatus comprising a light source producing light at a predetermined wavelength and power, and optical transmission means to train the light from the light source onto at least one external side of a tooth. Preferably, the optical transmission means comprises at least one optical guide. In a preferred embodiment, the optical transmission means comprises a pair of substantially parallel elongate light guides each light guide having a distal end provided with a reflective surface inclined at 45° to the direction of light transmission along the light guides, the reflective surfaces of the light guides being opposed such that light emitted from each light guide is parallel but opposite in direction. Preferably, the light source is a light emitting diode or array of light emitting diodes and the has a wavelength of from 550 to 690 nm, more preferably from 600 nm to 680 nm, even more preferably from 625 to 660 nm.

The present invention relates to photo-activated disinfection. In particular, the present invention relates to photo-activated disinfection in the oral cavity.

EP 637 976 describes the use of photosensitising compounds in killing microbes involved in a number of oral diseases by irradiation with laser light. The process involves gaining access to the treatment site, contacting the tissues wound or lesion with a photosensitising composition and irradiating the tissues, wound or lesion with laser light at a wavelength absorbed by the photosensitising composition.

For example, this process is taken further in our earlier patent application, WO00/62701 in which we describe a minimally invasive process and apparatus for treating dental caries. A small tunnel is prepared from the outer surface of the tooth to the site of the carious lesion. The carious dentine is then inoculated with a light photosensitising composition, such as those described in EP 637 976. An optical fibre is inserted into the tunnel. A proximal end of the fibre is coupled to a laser-light generator. The distal tip of the fibre is shaped to spread light around substantially the whole of the tooth cavity. As the photosensitising composition, we have preferred the use of toluidine blue O (TBO), requiring activation by laser light at a wavelength of 635 nm and with a power of approximately 100 mW.

This process has been found to be highly effective. However, sources of suitable laser light are expensive and there is a requirement for the light to be delivered close to the treatment site. It is with these disadvantages in mind that the present invention has been devised.

According to one aspect, the present invention provides a dental apparatus comprising a light source producing light at a predetermined wavelength and power, and optical transmission means to train the light from the light source onto at least one external side of a tooth.

Preferably, the optical transmission means comprises at least one optical guide.

More preferably, the optical transmission means comprises a pair of optical guides to train light onto two external sides of a tooth.

In the case of providing exposure to two external surfaces, each light guide preferably receives light from a respective light source. However, in alternative embodiments, for illuminating multiple surfaces the apparatus comprises a single light source together with means for dividing the light from such a source into a plurality of light guides.

In a preferred embodiment, the optical transmission means comprises a pair of substantially parallel elongate light guides each light guide having a distal end provided with a reflective surface inclined at 45° to the direction of light transmission along the light guides, the reflective surfaces of the light guides being opposed such that light emitted from each light guide is parallel but opposite in direction.

Suitably, the light source is a filtered white light source. Preferably, the light source is a light emitting diode or array of light emitting diodes. Preferably, the light has a wavelength of from 550 to 690 nm, more preferably from 600 nm to 680 nm, even more preferably from 625 to 660 nm.

In one embodiment, the apparatus further includes a supplementary light source and respective supplementary optical transmission means; wherein the supplementary optical transmission means is shaped and dimensioned for entry into a tooth cavity.

The present invention also provides a method for disinfection of the oral cavity or a wound or lesion in the oral cavity, the method comprising contacting the tissues, wound or lesion with a photosensitising composition, irradiating the tissues, wound or lesion with light at wavelength absorbed by the photosensitising composition.

The method is characterised in that the light source comprises a light emitting diode or an array of light emitting diodes.

Preferably, the tissues, wound or lesion are irradiated using the apparatus described above.

Preferably, the photosensitising composition comprises at least one photosensitiser selected from toluidine blue O, methylene blue, dimethylene blue or azure blue chloride. More preferably the photosensitiser is toluidine blue O. Most preferably, the sensitiser is toluidine blue O in the form of ‘tolonium chloride’, being the pharmaceutical grade of TBO wherein the purity and isomeric ratios are maintained.

Preferably, the light has a wavelength of from 550 to 690 nm, more preferably from 600 to 680 nm, even more preferably from 625 to 660 nm. Most preferably, the wavelength is about 660 nm or about 625 nm.

Preferably, the concentration of photosensitiser is in the region of 1 to 200 μg/ml.

The above and other aspects of the present invention will now be described in further detail, by way of example only, with reference to the accompanying drawings, in which:

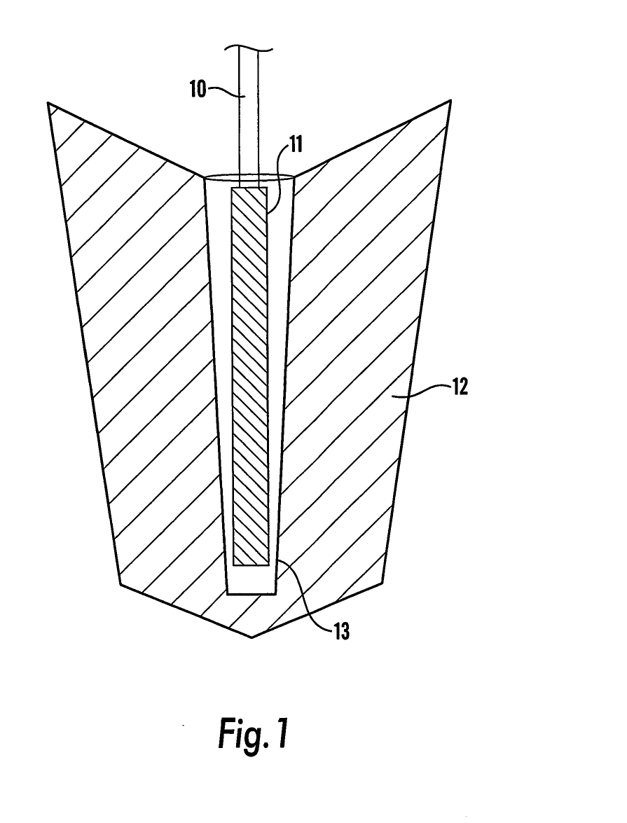

FIG. 1 is schematic sketch of a model used for calculation of absorbed energy density;

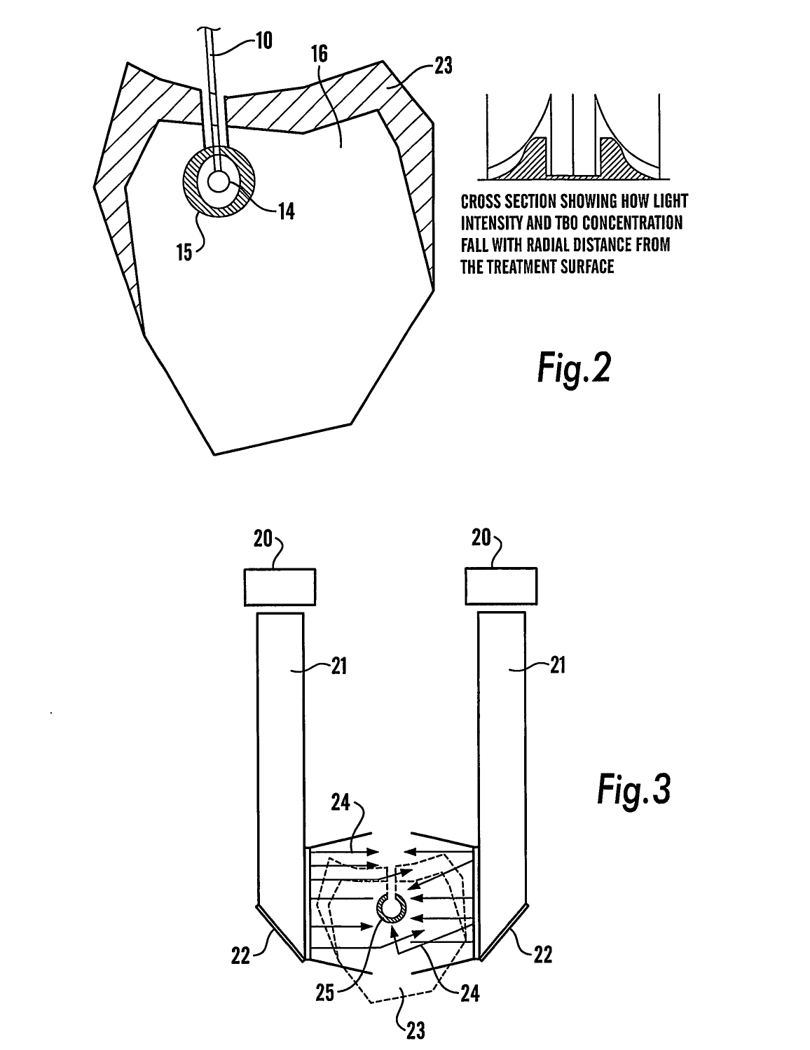

FIG. 2 is a schematic drawing illustrating the prior art method and apparatus of WO00/62701;

FIG. 3 is a schematic drawing of an apparatus in accordance with the present invention; and

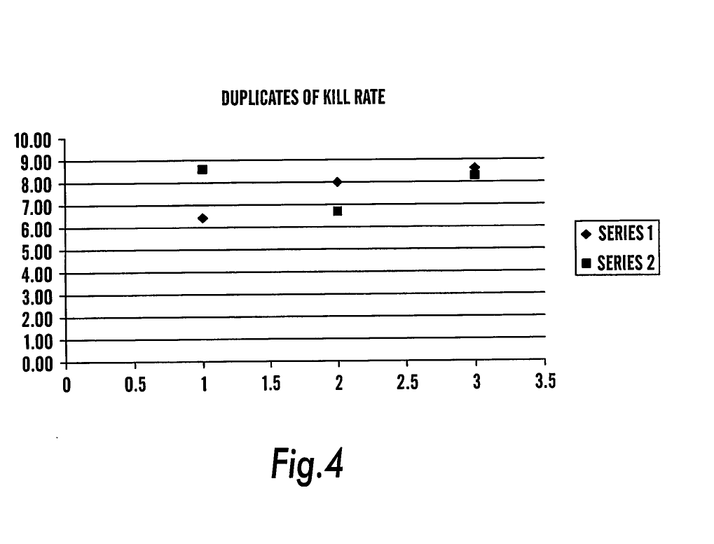

FIG. 4 is a chart illustrating bacterial kill with use of the apparatus of the present invention.

In this invention we have sought to use light emitting diodes (LEDs) because they are available as compact low-cost sources. They are available as multiplexed arrays, typically comprising 600 or more individual LEDs, with substantial output powers. The output wavelength spread of such devices, whilst not nearly as narrow as that of a laser, are still substantially narrower than any other parameter of significance such as the absorption profile of Toluidine Blue O (TBO) or variations in dental hard and soft tissue transmission.

The present invention will be illustrated with respect to the use of a solution of TBO as the photosensitiser composition. However, the principles as set out herein are equally applicable to other photosensitiser compositions.

The concentration of tolonium chloride will depend on the specific application and chosen wavelength. We have determined an optimum at 635 nm for caries of 12 μg/ml. Longer wavelengths and other applications especially periodontal disease where in vivo dilution may occur are likely to need greater concentrations, whereas shorter wavelengths may require reduced concentrations. Preferably, therefore, the concentration should fall within an overall range between 1 and 200 μg/ml.

Wanting to match the source wavelength with the maximum of the TBO absorption curve drove the choice of 635 nm for the laser used to activate the solution in the prior art. This would be the conventional teaching that would minimise the power needed from the source. In this invention we either chose the longer wavelength of around 660 nm or the shorter wavelength of around 625 nm. At the longer wavelength of 660 nm, a particular concentration of TBO has an absorption coefficient about one third that of the coefficient at 635 nm. The primary reason for this choice of wavelength is the availability of high power devices. Secondary advantages are in the improved transmission at this wavelength through dental hard tissue (an increase of around 5-10%) and, of special significance for the treatment of periodontal disease, improved transmission through the highly vascularised soft tissue of the guns (an increase of around 20-25%).

The alternative shorter wavelength of around 620 nm offers use of a match to a secondary maximum in the absorption of tolonium chloride and could be of particular value to the treatment of surface disease where normal tissue transmission is not a significant factor. In addition higher power devices are becoming available in this shorter wavelength band.

Laboratory studies have demonstrated, as would be expected, that achieving high bacterial kill, when using the optimum TBO concentration, is energy dependent. That is, the kill level is linearly related to the absorbed energy represented by a delivered power for a defined period. Although not directly studied, it is possible, given knowledge of the geometry of the treatment area and the size and shape of the emitter to estimate the critical parameter, namely the absorbed energy density (AED). That is, we know the AED to achieve 99.9% kill in, say, a root canal with 635 nm light delivered via a cylindrical emitter. (FIG. 1).

We can also note that an optimum concentration of TBO has been demonstrated. The reduction in efficacy as the concentration increases beyond the optimum is thought to be due to the accompanying increasing absorption limiting the light transmission through the treatment zone.

With this knowledge, calculations can be made to extrapolate to the situation envisaged in this invention.

FIG. 1 is a diagrammatic sketch of a model use for simple calculations of absorbed energy density for the arrangement described in WO00/62701. Light is transmitted from a laser (not shown) through an optical fibre 10 to an emitter 11. A tooth is represented at 12 in which a root canal 13 has been excavated. The emitter 11 is a cylinder roughly matching the size of a prepared root canal 13. The average diameter of the canal 13 is 0.8 mm and the length around 15 mm. The treatment area is therefore:

Area=πd L=0.4 cm²

High kills are achieved in this geometry using TBO on the inside surface of the root canal (with some penetration into the dentine), 100 mW power and 120 S exposure. Thus the total energy delivered is:

$$\begin{matrix}Energy ( at 635 nm ) delivered = Power \times Time \\ = 0.1 \times 120 \\ = 12 Joules\end{matrix}$$

The AED is then simply the energy delivered over the area exposed or:

AED=30 J/cm²

Moving to a wavelength of around 660 nm where the TBO absorption is one third its value at 635 nm implies that, for a similar efficacy, we would need to treble the ADE to 90 J/cm² if all other parameters remained the same. It is to be expected, however, operating at a wavelength having a lower absorption would allow a balancing increase in TBO concentration. If this change takes place then the AED (670) would be much the same as the AED (635). Although this is one route which could be pursued it is desirable but not necessary. Fundamentally one can always achieve the delivered dose by increasing the treatment duration. Clearly maintaining the AED at 30 J/cm² would be better from the operator's viewpoint.

In summary, from our investigations we have determined that a wavelength of around 660 nm can be used instead of 635 nm whilst maintaining the same AED by adjustment of the TBO concentration.

Laboratory studies to determine AED levels have been conducted in planktonic solutions of the sample bacterium ‘Streptococcus Mutans’. These indicate that, at 635 nm, the AED is in fact less than that calculated above and that highly effective kills of 7 to 8 orders of magnitude can be achieved at 15 J/cm². The experiments also provide evidence that, for thin layers where sensitiser absorption is not limiting efficacy, the AED is not reduced by increasing the sensitiser concentration. A summary of the preliminary results is shown in the graph attached as FIG. 4. The graph of FIG. 4 displays bacterial kill (in orders of magnitude) for three separate concentrations (between 10 and 50 μg/ml) of tolonium chloride. In all cases energy dose was 15 J/cm².

The process as envisaged by the WO0/62701 had an isotropic light emitter 14 in intimate contact with the treatment zone namely a carious lesion 15. It was viewed as treatment from “within” the lesion 15. The prior application of TBO would ensure that the residual tissue containing the causative bacteria took it up. FIG. 2 schematically illustrates this situation using a caries lesion model together with the likely distribution of both TBO and light intensity as a function of radial distance from the inner surface. The calculations of AED used the root canal model because the dentist prepares the canal to a standard size and thus makes the calculation more straightforward. A somewhat smaller average AED would apply in the caries model as evidenced by the demonstration of clinical efficacy using shorter exposure times.

From FIG. 2, it is apparent that “internal exposure” although clearly clinically efficacious does suffer from one feature namely the simultaneous drop in both local light intensity and TBO concentration. Thus there would be a clear advantage in the route described by the present invention that is illumination from outside the lesion, since this would give a more uniform value to the product of light intensity and TBO concentration. This would materially increase the depth of partially diseased tissue that could be effectively disinfected.

Finally, we calculate how much power is needed externally to the tooth in order to achieve the AED at a lesion situated in the centre of the tooth. An embodiment of the light delivery arrangement in accordance with the present invention is shown in FIG. 3. This also illustrates the exposed tooth and thus facilitates the necessary calculation.

Light from two LED arrays 20 is directly coupled to the proximal end of two respective optical guides of any suitable cross-section, typically square, rectangular or circular. These light guides, typically glass, are suitably around 5 mm in side dimension, matching closely the emitting area from the LEDs. At the distal end, a 45° reflective or mirrored surface 22 is provided such that the light is emitted substantially from one side of each guide. The two guides 21 are positioned, in use, on either side of a tooth 23.

The light emitted will enter the tooth 23 wherein light rays 24 will be transmitted and scattered to reach the centre. The scattering coefficient dominates the absorption coefficient at around 660 nm and so an observer at a lesion 25 would essentially see light from all directions. The calculation of the internal energy density is complicated but a simple estimate can be reached assuming pure scattering by “removing” the tooth and calculating the expanded beam area at the lesion position.

Hence, assuming a large tooth of 10 mm diameter with a lesion in the centre each emitting surface is 5 mm from the lesion, we will have an effective area of illumination at the lesion of 2.25 cm². Hence to achieve the maximum estimated AED required of 30 J/cm² we would need to deliver a total of 67.5 Joules. Allowing for a reduction in transmission of 75% through the tooth due to absorption, increases this figure to approximately 280 J or 140 J per light guide. Retaining 120 second illumination as a maximum equates to a light power of around 1 watt, a figure that is available from commercial LED arrays.

Clearly this is far in excess of that needed for the treatment of surface caries. Accordingly, the present invention provides a more convenient and more appropriate system in this case as well whether using sources around 625 nm or 660 nm.

To effectively treat periodontal disease, the apparatus can be an extension to that shown in FIG. 3. The use of a guide taper wherein one dimension of the square is reduced to 0.5 mm would allow its direct insertion into the periodontal pocket. Given that, at the wavelength of 660 nm, reasonable transmission through soft tissue can be achieved, illumination from the outside of the pocket would also be feasible. In either case a broad area delivery modality (as opposed to the point source of the prior art) would be advantageous.

To treat root canals effectively would require a delivery system that combined that shown in FIG. 3 together with a third smaller LED coupled via a taper to small diameter fibre. Although this would be not as inherently efficient, it would deliver light to the apex of the canal where external illumination would be least effective.

We propose here a new means by which the process of photo-activated bacterial killing may be realised. The new means is novel and involves an entirely different physical approach than those envisaged earlier.

Three distinguishing and novel features are:

i. The use of filtered white light sources or LED sources (Light Emitting Diodes) rather than lasers.

The use of longer wavelengths around 660 nm to both improve transmission through dentine and enamel or shorter wavelengths around 625 nm for surface disease and to take advantage of the availability of higher power devices.

ii. Applying the light externally to the tooth to circumvent the need to couple into small diameter fibres.
iii. The use of increased sensitiser concentrations (up t 200 μg/ml) to allow for both longer wavelength illumination and applications where significant in vivo dilution may occur.

1. A dental apparatus comprising a light source producing light at a predetermined wavelength and power, and optical transmission means to train, in use, the light from the light source onto at least one external side of a tooth.

2. An apparatus as claimed in claim 1 wherein the light source comprises a light emitting diode or array of light emitting diodes.

3. An apparatus as claimed in claim 1 wherein the light has a wavelength of from 550 to 690 nm.

4. An apparatus as claimed in claim 1 wherein the optical transmission means comprises at least one optical guide.

5. An apparatus as claimed in claim 4 wherein the optical transmission means comprises a pair of optical guides to train, in use, light onto two external sides of a tooth.

6. An apparatus as claimed in claim 5 wherein the apparatus comprises a single light source and further comprises light splitting means for dividing the light from the source into the pair of optical guides.

7. An apparatus as claimed in claim 5 wherein the optical transmission means comprises a pair of substantially parallel elongate optical guides each of said optical guides having a distal end provided with a reflective surface inclined at 45° to the direction of light transmission along the optical guides, the reflective surfaces of the optical guides being opposed such that light emitted from each light guide is parallel but opposite in direction.

8. An apparatus as claimed in claim 1 wherein the apparatus further includes a supplementary light source and respective supplementary optical transmission means; wherein the supplementary optical transmission means is shaped and dimensioned for entry into a tooth cavity.

9. A method for disinfection of the oral cavity or a wound or lesion in the oral cavity, the method comprising
contacting the tissues, wound or lesion with a photosensitising composition; and
irradiating the tissues, wound or lesion with light at wavelength absorbed by the photosensitizing composition; wherein
the light is provided by a light source comprising a light emitting diode or an array of light emitting diodes.

10. (canceled)

11. A method as claimed in claim 9 wherein the photo sensitising composition comprises at least one photosensitiser selected from the group consisting of: toluidine blue O, methylene blue, dimethylene blue and azure blue chloride.

12. A method as claimed in claim 11 wherein the photo sensitiser is toluidine blue O.

13. An apparatus as claimed in claim 1 wherein the light has a wavelength of from 600 nm to 680 nm.

14. An apparatus as claimed in claim 1 wherein the light has a wavelength of from 625 to 660 nm.

15. An apparatus as claimed in claim 2 wherein the optical transmission means comprises at least one optical guide.

16. An apparatus as claimed in claim 3 wherein the optical transmission means comprises at least one optical guide.

17. An apparatus as claimed in claim 2 wherein the apparatus further includes a supplementary light source and respective supplementary optical transmission means; wherein the supplementary optical transmission means is shaped and dimensioned for entry into a tooth cavity.

18. A method for disinfection of the oral cavity or a wound or lesion in the oral cavity, the method comprising:
contacting the tissues, wound or lesion with a photosensitising composition; and
irradiating the tissues, wound or lesion with light at wavelength absorbed by the photosensitizing composition; wherein
the light is provided by an apparatus comprising a light source producing the light at a predetermined wavelength and power, and optical transmission means to train, in use, the light from the light source onto at least one external side of a tooth.

19. The method of claim 18, wherein the light source comprises a light emitting diode or an array of light emitting diodes.

20. The method of claim 18, wherein the light has a wavelength of from 550 to 690 nm.

21. The method of claim 18, wherein the optical transmission means comprises at least one optical guide.
